(19) United States
(12) Patent Application Publication
(10) Pub. No.: US 2024/0180414 A1
(43) Pub. Date: Jun. 6, 2024

(54) INTRAVASCULAR IMAGING DEVICES

(71) Applicant: Boston Scientific Scimed, Inc., Maple Grove, MN (US)

(72) Inventors: Quinn Mackenzie Butler, Minneapolis, MN (US); Haruka Imura, Maple Grove, MN (US); Monica Ortiz Serrano, Maple Grove, MN (US); Andrew Strom, Golden Valley, MN (US); Joseph Alan Kronstedt, New Hope, MN (US); Jeffrey Adam Hammerstrom, New Hope, MN (US); Andrew Straw, Champlin, MN (US); Bryan Joseph Plunger, Shoreview, MN (US); Peter Phommahaxay, Maple Grove, MN (US)

(73) Assignee: Boston Scientific Scimed, Inc., Maple Grove, MN (US)

(21) Appl. No.: 18/526,283

(22) Filed: Dec. 1, 2023

Related U.S. Application Data
(60) Provisional application No. 63/429,404, filed on Dec. 1, 2022.

(51) Int. Cl. A61B 1/313; A61B 1/00; A61B 1/018

(52) U.S. Cl. CPC A61B 1/3137; A61B 1/0011; A61B 1/018; A61B 8/12

(57) ABSTRACT

Intravascular imaging devices and methods for making and using intravascular imaging devices are disclosed. An example intravascular imaging device may include a catheter shaft configured to be disposed within a blood vessel. The catheter shaft may have an imaging window region and an imaging lumen. The imaging lumen may be configured to accommodate and imaging assembly therein. A distal tip member may be coupled to the catheter shaft. The distal tip member may have a guidewire lumen formed therein. The imaging window region and the distal tip member may axially overlap.

### CROSS-REFERENCE TO RELATED APPLICATIONS

This application claims priority to U.S. Provisional Application Ser. No. 63/429,404, filed Dec. 1, 2022, the entirety of which is incorporated herein by reference.

### TECHNICAL FIELD

The present disclosure pertains to medical devices, and methods for manufacturing medical devices. More particularly, the present disclosure pertains to elongated intravascular imaging devices.

### BACKGROUND

A wide variety of medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An intravascular imaging device is disclosed. The intravascular imaging device comprises: a catheter shaft configured to be disposed within a blood vessel, the catheter shaft having an imaging window region and an imaging lumen; wherein the imaging lumen is configured to accommodate and imaging assembly therein; a distal tip member coupled to the catheter shaft, the distal tip member having a guidewire lumen formed therein; and wherein the imaging window region and the distal tip member axially overlap.

Alternatively or additionally to any of the embodiments above, the distal tip member includes a distal end region extending distally from the imaging window region and an overlap region where the imaging window region and the distal tip member axially overlap.

Alternatively or additionally to any of the embodiments above, the distal end region has a length of about 5-25 millimeters.

Alternatively or additionally to any of the embodiments above, the distal end region has a length of about 10-20 millimeters.

Alternatively or additionally to any of the embodiments above, the overlap region has a length of about 300 millimeters or less.

Alternatively or additionally to any of the embodiments above, the overlap region has a length of about 15-300 millimeters.

Alternatively or additionally to any of the embodiments above, the imaging lumen is in fluid communication with the guidewire lumen.

Alternatively or additionally to any of the embodiments above, the distal tip member has an axially-extending slit formed therein.

Alternatively or additionally to any of the embodiments above, the distal tip member has an axially-extending channel formed therein.

Alternatively or additionally to any of the embodiments above, the distal tip member and the imaging window region are integrally formed.

Alternatively or additionally to any of the embodiments above, the distal tip member and the imaging window region are formed from a single monolith of material.

Alternatively or additionally to any of the embodiments above, the distal tip member, the imaging window region, or both include a coating.

An intravascular imaging device is disclosed. The intravascular imaging device comprises: a catheter shaft assembly including a telescoping region and a catheter shaft extending distally therefrom; wherein the catheter shaft includes an imaging window region and a distal tip region having a guidewire lumen formed therein; wherein the distal tip region has a first portion disposed distally of the imaging window region and a second portion disposed along a side surface of the imaging window region; and an imaging core disposed within the catheter shaft assembly.

Alternatively or additionally to any of the embodiments above, the second portion has a length of about 300 millimeters or less.

Alternatively or additionally to any of the embodiments above, the second portion has a length of about 15-300 millimeters.

Alternatively or additionally to any of the embodiments above, the imaging window region has an imaging lumen formed therein that is in fluid communication with the guidewire lumen.

Alternatively or additionally to any of the embodiments above, the distal tip region has an axially-extending slit formed therein.

Alternatively or additionally to any of the embodiments above, the distal tip region has an axially-extending channel formed therein.

Alternatively or additionally to any of the embodiments above, the distal tip region and the imaging window region are integrally formed.

Alternatively or additionally to any of the embodiments above, the distal tip region and the imaging window region are formed from a single monolith of material.

Alternatively or additionally to any of the embodiments above, the distal tip member, the imaging window region, or both include a coating.

A method for imaging a blood vessel is disclosed. The method comprises: disposing an intravascular imaging device within a blood vessel, the intravascular imaging device comprising: a catheter shaft assembly including a telescoping region and a catheter shaft extending distally therefrom, wherein the catheter shaft includes an imaging window region and a distal tip region having a guidewire lumen formed therein, wherein the distal tip region has a first portion disposed distally of the imaging window region and a second portion disposed along a side surface of the imaging window region, and an imaging core disposed within the catheter shaft assembly; and translating the imaging core relative to the catheter shaft assembly.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of an example medical device.

FIG. 2 is a side view of another illustrative medical device.

FIG. 3 is a perspective view of the medical device of FIG. 2 in a retracted configuration.

FIG. 4 is a perspective view of the medical device of FIG. 2 in an extended configuration.

FIG. 5 is a cross-sectional side view of a portion of an example medical device.

FIG. 6 is a cross-sectional view taken through line 6-6 in FIG. 5.

FIG. 7 is a cross-sectional view taken through line 7-7 in FIG. 5.

FIGS. 7A-7C are alternative cross-sectional views taken through line 7-7 in FIG. 5.

FIG. 8 is an alternative cross-sectional view of an example medical device.

FIG. 9 is an alternative cross-sectional view of an example medical device.

FIG. 10 is an alternative cross-sectional view of an example medical device.

FIG. 11 is a cross-sectional side view of a portion of an example medical device.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “of” is generally employed in its sense including “and/of” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

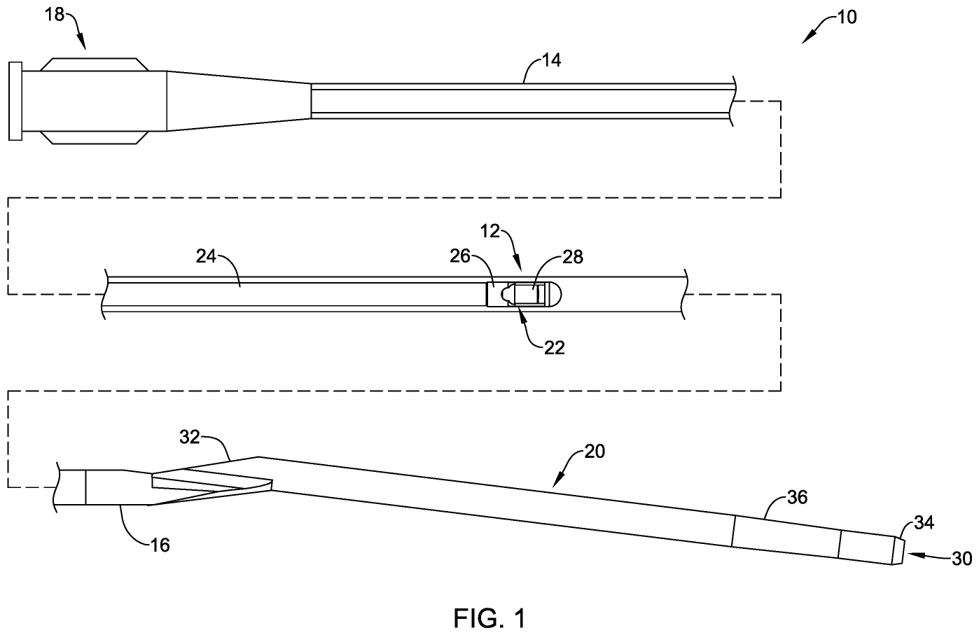

FIG. 1 is a side view of an example medical device 10. In at least some instances, the medical device 10 takes the form of an imaging medical device. For example, the medical device 10 may be an intravascular ultrasound (IVUS) device that may be used to image a blood vessel. The structure/form of the medical device 10 can vary. In some instances, the medical device 10 may include a catheter shaft 12 having a proximal end region 14 and a distal end region 16. A proximal hub or connector 18 may be coupled to or otherwise disposed adjacent to the proximal end region 14. A tip member may be coupled to or otherwise disposed adjacent to the distal end region 16. The tip member 20 may include a guidewire lumen 30 having a guidewire exit port 32, an atraumatic distal end 34, one or more radiopaque markers 36, and/or other features. In some embodiments, the tip member 20 may extend at a non-parallel angle to the proximal end region 14 of the catheter shaft 12. An imaging assembly 22 may be disposed within a lumen of the catheter shaft 12. In general, the imaging assembly may be used to capture/generate images of a blood vessel. In some instances, the medical device may include devices and/or features similar to those disclosed in U.S. Patent Application Pub. No. US 2012/0059241 and U.S. Patent Application Pub. No. US 2017/0164925, the entire disclosures of which are herein incorporated by reference. In at least some instances, the medical device 10 may resemble and/or include features that resemble the OPTICROSS™ Imaging Catheter, commercially available from BOSTON SCIENTIFIC, Marlborough, MA.

The imaging assembly 22 may include a drive cable or shaft 24, a housing 26, and an imaging member or transducer 28 coupled to the drive cable 24 and/or housing 26. In at least some instances, the transducer 28 includes an ultrasound transducer. Other transducers are also contemplated. The transducer 28 may be rotatable and/or axially translatable relative to the shaft 12. For example, the drive cable 24 may be rotated and/or translated in order to rotate and/or translate the transducer 28 (and the housing 26).

While not explicitly shown in FIG. 1, the medical device 10 may include a telescoping assembly, configured to allow the medical device operator to move the drive shaft 24 including the imaging assembly 22 proximally and distally within the catheter shaft 12, without having to move the entire catheter within the patient. This allows the catheter operator to easily change the location of the imaging assembly or other medical device within the patient. For example, the telescoping section may be actuated to change the location of the imaging assembly 22 within the catheter shaft 12.

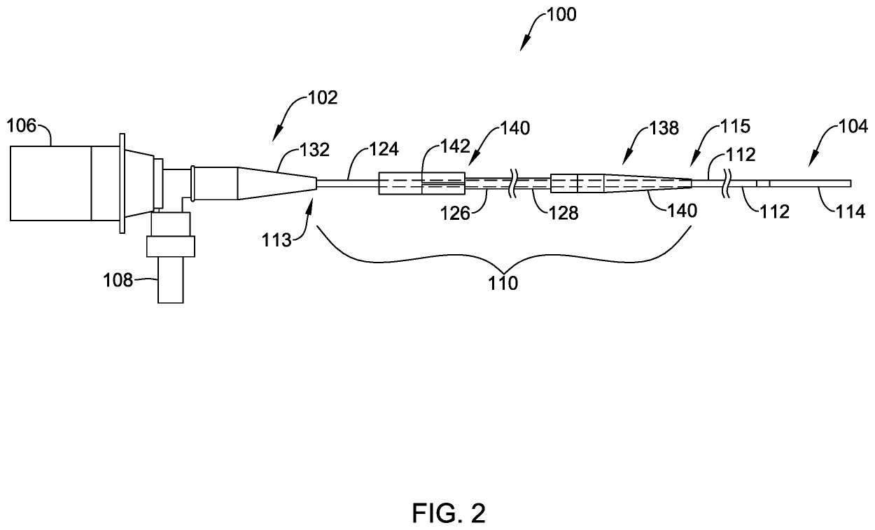

FIG. 2 illustrates a side view of another example medical device 100 (e.g., an intravascular imaging device 100) that may be similar in form and function to other medical devices disclosed herein. The medical device 100 extends from a proximal end region 102 to a distal end region 104. A proximal hub 106 may be affixed adjacent to the proximal end region 102. The proximal hub 106 may include a check valve and flush port 108. In order to flush the medical device 100, fluid may be infused at the flush port 108. The medical device 100 may further include a telescoping section 110 extending from a proximal end region 113 to a distal end region 115 and positioned between the proximal end region 102 and the distal end region 104 of the medical device 100. An elongate shaft or catheter 112 extends distally from the distal end region 115 of the telescoping section 110. The elongate shaft 112 may include a tip member 114 adjacent the distal end region 104 of the medical device 100. The tip member 114 may be similar in form and function to the tip member 20 described with respect to FIG. 1. For example, the tip member 114 may include a guidewire lumen having a guidewire exit port, an atraumatic distal end, one or more radiopaque markers, and/or other features.

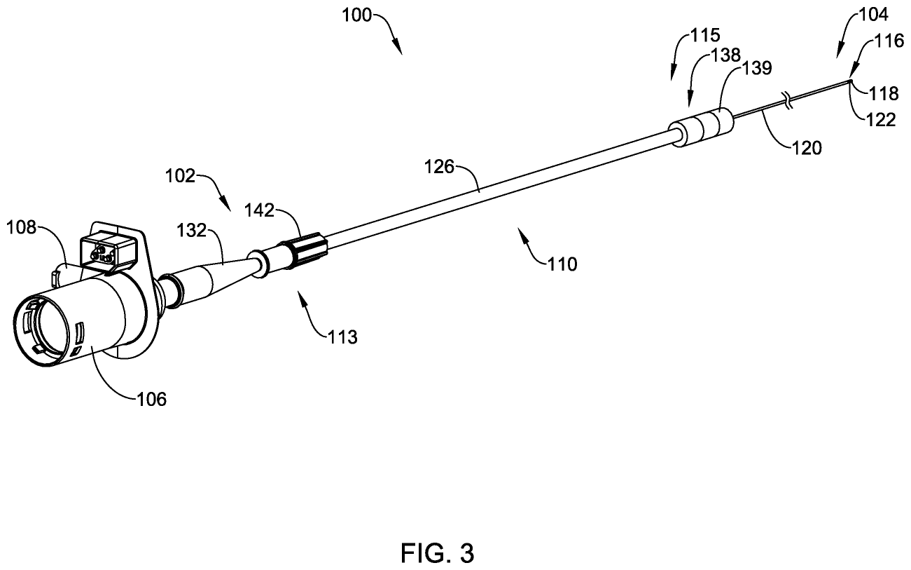

An imaging assembly 116 (see, for example, FIG. 3) may be movably positioned within a lumen of the elongate shaft 112. The imaging assembly 116 may include a drive cable or shaft 120, a housing 122, and an imaging member or transducer 118 coupled to the drive cable 120 and/or housing 122. It is contemplated that the imaging assembly 116 may include or be replaced with another medical device, such as, but not limited to, a cutting head, or other device. The particular device chosen for the drive cable 120 may be selected based on the desired function for the medical device 100. The drive cable 120 may extend proximally from the imaging member 118 through the telescoping section 110 to the proximal hub 106. The proximal hub 106 may contain components adapted to interface the drive cable 120 with a power source and/or other electronic couplings. In some cases, a proximal end of the drive cable 120 may be affixed to the proximal hub 106. While not explicitly shown, the drive cable 120 may include a single layer outer jacket or coating or a two-layer outer jacket or coating, as desired. If so provided, the outer jacket may extend a full length of the drive cable 120 or less than a full length of the drive cable 120.

The telescoping section 110 may include a first or intermediate sheath 124, a second or outer sheath 126, and a third or inner sheath 128. Generally, the outer sheath 126 may be disposed over the intermediate sheath 124 and the intermediate sheath 124 disposed over the inner sheath 128. The intermediate sheath 124 may be axially and/or rotatably displaced relative to the outer and inner sheaths 126, 128 such that movement of the proximal hub 106 is translated to movement of the intermediate sheath 124 and the drive cable 120. A distal hub 138 may be positioned adjacent the distal end region 115 of the telescoping section 110. The distal hub 138 may include a distal strain relief 139 configured to be coupled to the elongate shaft 112. Further, the distal ends of the outer sheath 126 and the inner sheath 128 may each be fixedly secured to the distal hub 138.

The intermediate sheath 124 extends distally from a proximal end region 130 coupled to a proximal strain relief 132 to a distal end 134 extending within the outer sheath 126. The intermediate sheath 124 may have a constant diameter from the proximal end region 130 to the distal end 134, although this is not required. The proximal strain relief 132 is coupled to the proximal hub 106. The intermediate sheath 124 is movable relative to the inner and outer sheaths 128, 126 such that the distal end 134 of the intermediate sheath 124 is movable between the distal hub 138 and a housing 142. The intermediate sheath 124 defines a lumen extending from the proximal end region 130 to the distal end 134 thereof. The lumen may receive and/or house a portion of the drive shaft 120 and/or the inner sheath 128.

The outer sheath 126 extends distally from a housing or receptacle 142 to a distal end (e.g., which may be affixed to the distal hub 138). The outer sheath 126 defines a lumen extending from the proximal end region to the distal end. The lumen may receive or house a portion of the inner sheath 128 and/or the intermediate sheath 124.

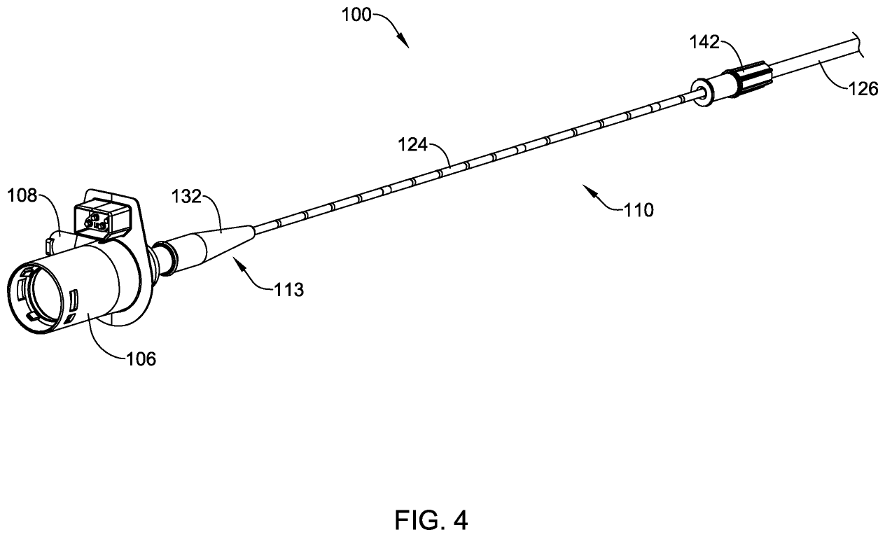

The inner sheath 128 extends distally from a proximal end region to a distal end affixed to the distal hub. The inner sheath 128 defines a lumen extending from the proximal end region to the distal end. The lumen may receive or house a portion of the drive shaft 120. For example, the inner sheath 128 may be configured to support the drive shaft 120 when the intermediate sheath 124 is in a proximally displaced configuration (see, for example, FIG. 4). In some embodiments, the proximal end region of the inner sheath 128 may be positioned adjacent to the proximal end region of the outer sheath 126. In other embodiments, the proximal end region of the inner sheath 128 may be distal to the proximal end region of the outer sheath 126.

FIG. 3 illustrates a perspective view of the medical device 100 of FIG. 2 with the proximal hub 106 and the intermediate sheath 124 (and hence the drive shaft 120) in a distalmost position. This configuration may be considered to be fully retracted, as the medical device 100 has the shortest length. In FIG. 3, the elongate shaft 112 is not shown to more clearly show the structure of the imaging assembly 116. In the embodiment of FIG. 3, the intermediate sheath 124 has been distally advanced within the lumen of the outer sheath 126. Distal movement of the intermediate sheath 124 may be limited by a mechanical stop created between the proximal strain relief 132 and the housing 142. When the proximal hub 106 and the intermediate sheath 124 are in a distalmost position, a majority of the length of the lumen 136 of the intermediate sheath 124 may surround the inner sheath 128.

FIG. 4 illustrates a perspective view of a proximal portion of the medical device 100 of FIG. 2 with the proximal hub 106 and the intermediate sheath 124 (and hence the drive shaft 120) near a proximal most position. This configuration may be considered to be fully extended, as the medical device 100 has the greatest length. In the embodiment of FIG. 4, the intermediate sheath 124 has been proximally displaced within the lumen of the outer sheath 126. Proximal movement of the intermediate sheath 124 may be limited by a mechanical stop created between mating features on a distal end region of the intermediate sheath 124 and the housing 142. When the proximal hub 106 and the intermediate sheath 124 are in a proximal most position, a majority of the length of the lumen 136 of the intermediate sheath 124 may surround the drive cable 120.

While FIGS. 3 and 4 illustrate the approximate extremes of the movement of the telescoping section 110, the proximal hub 106 and the intermediate sheath 124 may be positioned at any location between. As the drive cable 120 is coupled to the proximal hub 106, proximal and distal movement is translated to the drive cable 120 and the imaging assembly 116 to allow the imaging assembly to move without moving the entire medical device 100. It is further contemplated that rotational movement of the proximal hub 106 will also be translated to the drive shaft 120 and imaging assembly 116 to allow for rotation of the imaging assembly 116 within the elongate shaft 112.

During a coronary intervention, an imaging device may be navigated through the tortuous anatomy. When doing so, it is possible that the imaging device may kink or otherwise deform in a way that may disrupt the function of the device. Disclosed herein are intravascular imaging devices that include structural features to the device and that, for example, may help to reduce kinking as well as provide additional desirable benefits.

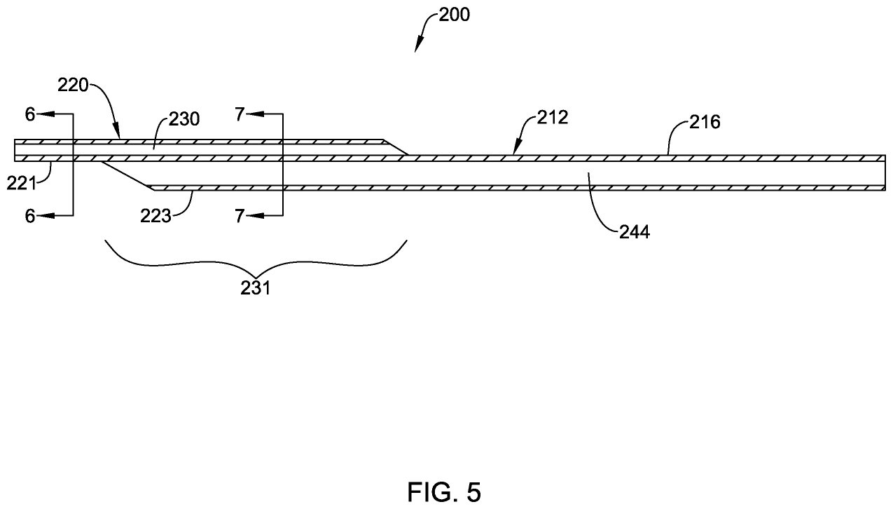

FIG. 5 is a side view of a portion of another example medical device 200 (e.g., an intravascular imaging device 200) similar in form and function to those disclosed herein. Here, only a distal portion 216 of an elongate shaft 212 is shown. The elongate shaft 212 may have an imaging lumen 244 formed therein that may, for example, accommodate an imaging assembly or imaging core (not shown in FIG. 5, but may be similar in form and function to the imaging assembly 116 disclosed herein) to be disposed therein. The elongate shaft 212 may include an imaging window region 223. In at least some instances, the imaging window region 223 may be adjacent to the distal end region of the elongate shaft 212 and may allow for an imaging assembly disposed in the imaging lumen 244 to image a blood vessel. It can be appreciated that the medical device 200 may also include a telescoping section (not shown in FIG. 5, but may be similar in form and function to the telescoping section 110 disclosed herein).

The elongate shaft 212 may also include a distal tip member or distal tip region 220. The distal tip member 220 may be similar in form and function to the distal tip member 20. For example, the distal tip member 220 may include a guidewire lumen 230. In some instances, the distal tip member 220 may be a separate tube or shaft that is coupled to the elongate shaft 212. In other instances, the elongate shaft 212 and the distal tip member 220 may be integrally formed and/or formed from a single monolith of material. For example, the elongate shaft 212 and the distal tip member 220 may be formed (e.g., as singular structure) by molding or extrusion. Other processes and arrangements are contemplated.

As suggested herein, it may desirable to provide structural support to the elongate shaft 212 so as to provide improved pushability, less kinking, and other desirable benefits. In some instances, the structural support may be added, at least in part, by axially overlapping at least a portion of the distal tip member 220 with the elongate shaft 212 (e.g., axially overlapping the distal tip member 220 with the imaging window region 223). In FIG. 5, the overlapping region is marked by reference number 231. The overlapping region 231 may have length that corresponds to the length of the imaging window. In some of these and in other instances, the overlapping region 231 may have a length of about 400 millimeters or less, or about 300 millimeters or less, or about 15-300 millimeters. These lengths are just examples. Other lengths are contemplated. In some instances, the entire distal tip member 220 may overlap with the elongate shaft 212. In other instances, a distal end region 221 of the distal tip member 220 may extend distally from the elongate shaft 212 (and/or the distal end region 221 of the distal tip member 220 may extend distally from the imaging window region 223). The distal end region 221 may have a length of about 5-millimeters, or about 10-20 millimeters, or about 15 millimeters. These lengths are just examples. Other lengths are contemplated.

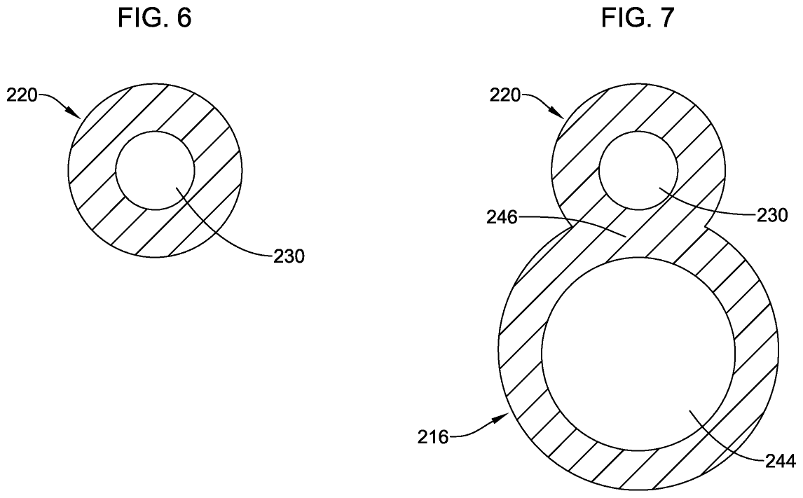

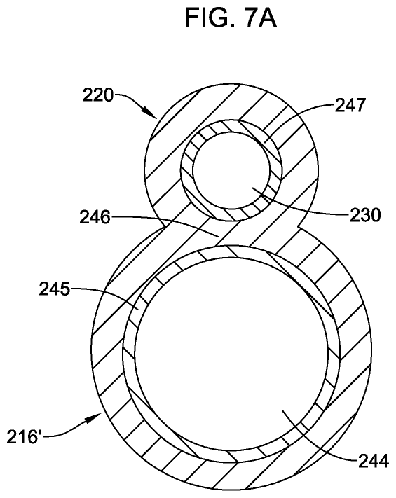

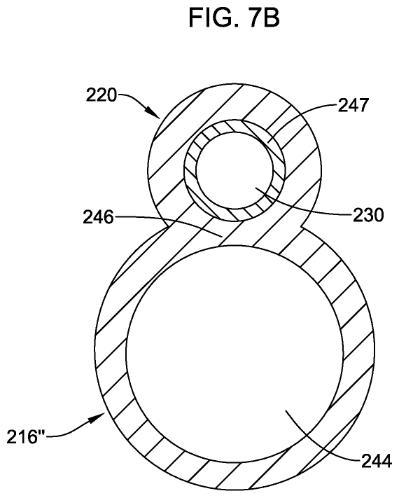

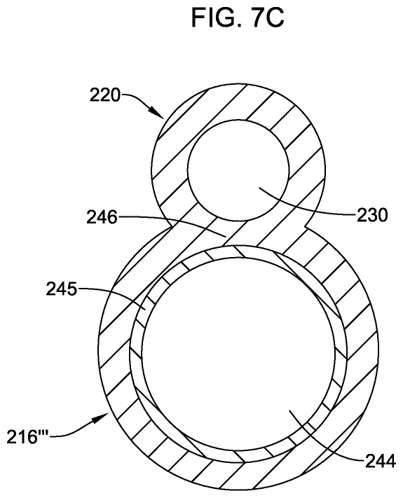

FIGS. 6-7 are cross-sectional view of the elongate shaft 212 and/or the distal tip member 220. For example, FIG. 6 is a cross-sectional view depicting the distal tip member 220 and showing the guidewire lumen 230. FIG. 7 is a cross-sectional view depicting the distal tip member 220, the guidewire lumen 230, the distal portion 216 (e.g., of the elongate shaft 212), and the imaging lumen 244. Also depicted is a wall or region 246 generally disposed between the guidewire lumen 230 and the imaging lumen 244. In some instances, the wall 246 may separate the guidewire lumen 230 and the imaging lumen 244. Other shafts are contemplated such as the elongate shaft 312 shown in FIG. 8. Here, adjacent the distal portion 316 of the elongate shaft 312, at least a portion of the wall (e.g., the wall 246) is removed/absent such that the imaging lumen 344 and the guidewire lumen 330 (e.g., formed in the distal tip member 320) are in fluid communication with one another.

In some instances, it may be desirable to add a lubricous liner or coating to the elongate shaft 212, for example along the guidewire lumen 230, the imaging lumen 244, or both. For example, FIG. 7A is an alternative distal portion 216′ of the elongate shaft 212 where a first liner or coating 245 is disposed along the imaging lumen 244 and a second liner or coating 247 is disposed along the guidewire lumen 230. Suitable materials for the coatings 245, 247 may include hydrophilic materials, ultra-high molecular weight (UHMW) polyethylene, high-density polyethylene (HDPE), a polyamide (e.g., such as polyamide 66 or PA66), a nylon (e.g., such as nylon 66), silicone, polytetrafluoroethylene (PTFE), ethylene tetrafluoroethylene (ETFE), polyetheretherketone (PEEK), polyimide (PI), acetal, polyethylene terephthalate (PET), fluorinated ethylene propylene (FEP), combinations thereof, and/or the like. In some instances, the coatings 245, 247 may be directly attached to a surface of the elongate shaft 212. In other instances, an intermediate or tie layer may be disposed between the elongate shaft 212 and the coatings 245, 247.

As shown in FIG. 7A, the coatings 245, 247 may be along both the imaging lumen 244 and the guidewire lumen 230. In other instances, the coating 247 may be disposed along the guidewire lumen 230 (e.g., of an alternative distal portion 216″ of the elongate shaft 212 with only the coating 247 disposed along the guidewire lumen 230) as shown in FIG. 7B. In still other instances, the coating 245 may be disposed along the imaging lumen 244 (e.g., of an alternative distal portion 216′″ of the elongate shaft 212 with only the coating 245 disposed along the imaging lumen 244) as shown in FIG. 7C.

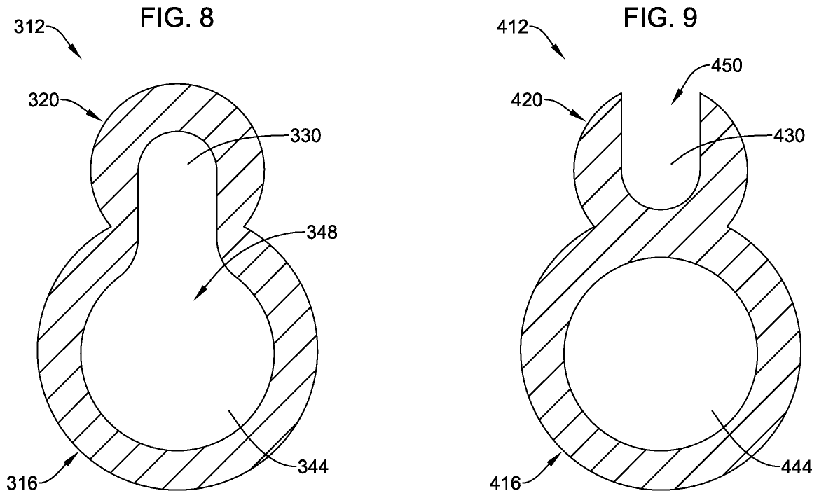

FIG. 9 illustrates a portion of an example elongate shaft 412 that may be similar in form and function to other shafts disclosed herein. The distal portion 416 of the elongate shaft 412 is shown along with the imaging lumen 444. Also shown is the distal tip member 420 with the guidewire lumen 430 formed therein. An axially-extending slot 450 may be formed in the distal tip member 420. The axially-extending slot 450 may remove at least some of the material of the distal tip member 420. This may at least partially reduce some of the impact of the distal tip member 420 on imaging. For example, by forming the axially-extending slot 450 in the distal tip member 420, there may be less structure/material along the imaging pathway so that it may be more efficient for an imaging assembly to image through the imaging window of the elongate shaft 412 and through the distal tip member 420. In some instances, the axially-extending slot 450 may be described as a channel, a “U-shaped” channel, or the like.

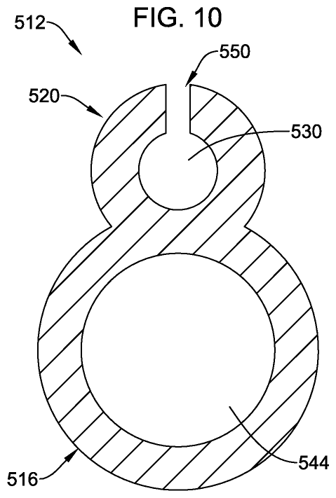

FIG. 10 illustrates a portion of an example elongate shaft 512 that may be similar in form and function to other shafts disclosed herein. The distal portion 516 of the elongate shaft 512 is shown along with the imaging lumen 544. Also shown is the distal tip member 520 with the guidewire lumen 530 formed therein. An axially-extending slit 550 may be formed in the distal tip member 520. The axially-extending slit 550 may remove at least some of the material of the distal tip member 520. This may at least partially reduce some of the impact of the distal tip member 520 on imaging. For example, by forming the axially-extending slit 550 in the distal tip member 520, there may be less structure/material along the imaging pathway so that it may be more efficient for an imaging assembly to image through the imaging window of the elongate shaft 512 and through the distal tip member 520. In some instances, the axially-extending slot 450 may be described as a channel, a “C-shaped” channel, or the like.

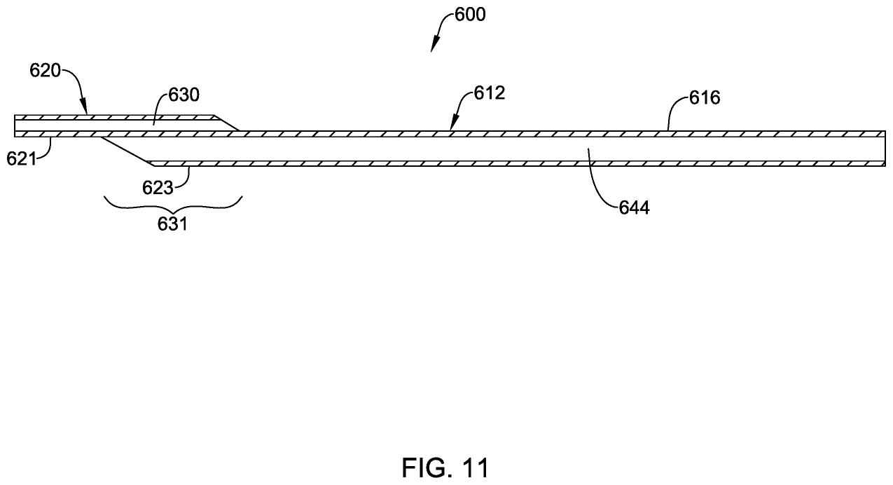

FIG. 11 is a side view of a portion of another example medical device 600 (e.g., an intravascular imaging device 600) similar in form and function to those disclosed herein. Here, only a distal portion 616 of an elongate shaft 612 is shown. The elongate shaft 612 may have an imaging lumen 644 formed therein that may, for example, accommodate an imaging assembly or imaging core. The elongate shaft 612 may include an imaging window region 623. In at least some instances, the imaging window region 623 may be adjacent to the distal end region of the elongate shaft 612 and may allow for an imaging assembly disposed in the imaging lumen 644 to image a blood vessel. The elongate shaft 612 may also include a distal tip member or distal tip region 620. The distal tip member 620 may be similar in form and function to the distal tip member 20. For example, the distal tip member 620 may include a guidewire lumen 630.

In some instances, at least a portion of the distal tip member 620 with the elongate shaft 612 (e.g., axially overlapping the distal tip member 620 with the imaging window region 623). In FIG. 11, the overlapping region is marked by reference number 631. The overlapping region 631 may have length that corresponds to the length of the imaging window. In some of these and in other instances, the overlapping region 631 may have a length that is shorter than the overlapping region 231 (e.g., depicted in FIG. 5). For example, the overlapping region 631 may have a length of about 200 millimeters or less, or about 100 millimeters or less, or about 15-100 millimeters. In some instances, the entire distal tip member 620 may overlap with the elongate shaft 612. In other instances, a distal end region 621 of the distal tip member 620 may extend distally from the elongate shaft 612 (and/or the distal end region 621 of the distal tip member 620 may extend distally from the imaging window region 623).

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An intravascular imaging device, comprising:
a catheter shaft configured to be disposed within a blood vessel, the catheter shaft having an imaging window region and an imaging lumen;
wherein the imaging lumen is configured to accommodate and imaging assembly therein;
a distal tip member coupled to the catheter shaft, the distal tip member having a guidewire lumen formed therein; and
wherein the imaging window region and the distal tip member axially overlap.

2. The intravascular imaging device of claim 1, wherein the distal tip member includes a distal end region extending distally from the imaging window region and an overlap region where the imaging window region and the distal tip member axially overlap.

3. The intravascular imaging device of claim 2, wherein the distal end region has a length of about 5-25 millimeters.

4. The intravascular imaging device of claim 2, wherein the distal end region has a length of about 10-20 millimeters.

5. The intravascular imaging device of claim 2, wherein the overlap region has a length of about 300 millimeters or less.

6. The intravascular imaging device of claim 2, wherein the overlap region has a length of about 15-300 millimeters.

7. The intravascular imaging device of claim 1, wherein the imaging lumen is in fluid communication with the guidewire lumen.

8. The intravascular imaging device of claim 1, wherein the distal tip member has an axially-extending slit formed therein.

9. The intravascular imaging device of claim 1, wherein the distal tip member has an axially-extending channel formed therein.

10. The intravascular imaging device of claim 1, wherein the distal tip member and the imaging window region are integrally formed.

11. The intravascular imaging device of claim 1, wherein the distal tip member, the imaging window region, or both include a coating.

12. An intravascular imaging device, comprising:
a catheter shaft assembly including a telescoping region and a catheter shaft extending distally therefrom;
wherein the catheter shaft includes an imaging window region and a distal tip region having a guidewire lumen formed therein;
wherein the distal tip region has a first portion disposed distally of the imaging window region and a second portion disposed along a side surface of the imaging window region; and
an imaging core disposed within the catheter shaft assembly.

13. The intravascular imaging device of claim 12, wherein the second portion has a length of about 300 millimeters or less.

14. The intravascular imaging device of claim 12, wherein the second portion has a length of about 15-300 millimeters.

15. The intravascular imaging device of claim 12, wherein the imaging window region has an imaging lumen formed therein that is in fluid communication with the guidewire lumen.

16. The intravascular imaging device of claim 12, wherein the distal tip region has an axially-extending slit formed therein.

17. The intravascular imaging device of claim 12, wherein the distal tip region has an axially-extending channel formed therein.

18. The intravascular imaging device of claim 12, wherein the distal tip region and the imaging window region are integrally formed.

19. The intravascular imaging device of claim 12, wherein the distal tip region and the imaging window region are formed from a single monolith of material.

20. A method for imaging a blood vessel, the method comprising:
disposing an intravascular imaging device within a blood vessel, the intravascular imaging device comprising:
a catheter shaft assembly including a telescoping region and a catheter shaft extending distally therefrom,
wherein the catheter shaft includes an imaging window region and a distal tip region having a guidewire lumen formed therein,
wherein the distal tip region has a first portion disposed distally of the imaging window region and a second portion disposed along a side surface of the imaging window region, and
an imaging core disposed within the catheter shaft assembly; and
translating the imaging core relative to the catheter shaft assembly.
